(19) United States
(12) Patent Application Publication
(10) Pub. No.: US 2003/0187430 A1
(43) Pub. Date: Oct. 2, 2003

(54) System and method for measuring power at tissue during RF ablation

(76) Inventor: James C. Vorisek, Riverside, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/098,951

(22) Filed: Mar. 15, 2002

(51) Int. Cl. A61B 18/14

(52) U.S. Cl. 606/34; 606/41

(57) ABSTRACT

An electrode and a voltage-measurement reference device are adapted to be positioned relative to a tissue load such that the load is generally located between the electrode and the reference device. A first wire and a second wire are electrically connected to the electrode. A power control system delivers RF current to the load through the first wire and measures the voltage across the load between the second wire and the reference device. The power control system measures the RF current through the first wire and determines the power delivered to the load using the measured current and voltage. The first and second wires function as thermocouple leads which, in combination with the electrode to which they are attached, form a thermocouple. The power control system monitors the voltage across the leads and determines the temperature at the electrode either during the delivery of current or alternatively, when current is not being delivered.

### BACKGROUND OF THE INVENTION

1. Field of the Invention

The invention relates generally to an electrophysiological (“EP”) catheter system and method for ablating biological tissue within a biological site, and more particularly to an EP system and method for determining the amount of RF power delivered to biological tissue during ablation.

The systems and methods of the invention are used during RF ablation procedures and thus involve the use of devices which operate to produce and measure signals in the RF range. For ease in describing the invention, the “RF” nomenclature is sometimes not used in the specification with respect to often repeated terms such as RF current, RF voltage, RF power, etc.

2. Description of the Related Art

The heart beat in a healthy human is controlled by the sinoatrial node (“SA node”) located in the wall of the right atrium. The SA node generates electrical signal potentials that are transmitted through pathways of conductive heart tissue in the atrium to the atrioventricular node (“AV node”) which in turn transmits the electrical signals throughout the ventricle by means of the His and Purkinje conductive tissues. Improper growth, remodeling, or damage to the conductive tissue in the heart can interfere with the passage of regular electrical signals from the SA and AV nodes. Electrical signal irregularities resulting from such interference can disturb the normal rhythm of the heart and cause an abnormal rhythmic condition referred to as “cardiac arrhythmia.”

While there are different treatments for cardiac arrhythmia, including the application of anti-arrhythmia drugs, in many cases ablation of the damaged tissue can restore the correct operation of the heart. Such ablation can be performed percutaneously, i.e., a procedure in which a catheter is introduced into the patient through an artery or vein and directed to the atrium or ventricle of the heart to perform single or multiple diagnostic, therapeutic, and/or surgical procedures. In such case, an ablation procedure is used to destroy the tissue causing the arrhythmia in an attempt to remove the electrical signal irregularities or create a conductive tissue block to restore normal heart beat. Successful ablation of the conductive tissue at the arrhythmia initiation site usually terminates the arrhythmia or at least moderates the heart rhythm to acceptable levels. A widely accepted treatment for arrhythmia involves the application of RF energy to the conductive tissue.

In the case of atrial fibrillation (“AF”), a procedure published by Cox et al. and known as the “Maze procedure” involves the formation of continuous atrial incisions that prevent atrial reentry and allow sinus impulses to activate the entire myocardium. While this procedure has been found to be successful, it involves an intensely invasive approach. It is more desirable to accomplish the same result as the Maze procedure by use of a less invasive approach, such as through the use of an appropriate EP catheter system providing RF ablation therapy. In ablation therapy, transmural lesions are formed in the atria to prevent atrial reentry and to allow sinus impulses to activate the entire myocardium. In this sense transmural is meant to include lesions that pass through the atrial wall or ventricle wall from the interior surface (endocardium) to the exterior surface (epicardium).

There are two general methods of applying RF energy to cardiac tissue, unipolar and bipolar. In the unipolar method a large surface area electrode; e.g., a backplate, is placed on the chest, back or other external location of the patient to serve as a return. The backplate completes an electrical circuit with one or more electrodes that are introduced into the heart, usually via a catheter, and placed in intimate contact with the aberrant conductive tissue. In the bipolar method, electrodes introduced into the heart have different potentials and complete an electrical circuit between themselves. In both the unipolar and the bipolar methods, the RF current traveling between the electrodes of the catheter and between the electrodes and the backplate enters the tissue and induces a temperature rise in the tissue resulting in the creation of ablation lesions.

During ablation, RF energy is applied to the electrodes to raise the temperature of the target tissue to a lethal, non-viable state. In general, the lethal temperature boundary between viable and non-viable tissue is between approximately 45° C. to 55° C. and more specifically, approximately 48° C. Tissue heated to a temperature above 48° C. for several seconds becomes permanently non-viable and defines the ablation volume. Tissue adjacent to the electrodes delivering RF energy is heated by resistive heating which is conducted radially outward from the electrode-tissue interface. The goal is to elevate the tissue temperature, which is generally at 37° C., fairly uniformly to an ablation temperature above 48° C., while keeping both the temperature at the tissue surface and the temperature of the electrode below 100° C. In clinical applications, the target temperature is set below 70° C. to avoid coagulum formation.

In order to avoid excessive delivery of RF energy and possible tissue dessication often associated with such energy delivery, it is desirable to monitor the amount of RF power being delivered to the tissue through the electrodes. In known ablation systems this is done by monitoring the RF voltage and the RF current of the power output directly at the output of the power amplifier at a location within the RF generator as shown in FIG. 10. The voltage and current measurements are then used to determine the power being delivered. However, RF voltage measurements taken at the output of the power amplifier are somewhat inaccurate because losses from transmission lines, i.e., electrode leads, or other inductively or capacitively reactive elements are not taken into account. Inaccuracy in the RF voltage measurement, in turn, leads to inaccuracy in the RF power measurement, thus rendering the power monitoring features of these systems somewhat ineffective.

Hence, those skilled in the art have recognized a need for a RF power delivery monitoring system and method that generally avoids the power-measurement inaccuracies caused by transmission lines and reactive elements to thereby provide a more accurate measurement of the RF power being delivered to the tissue during RF ablation. The invention fulfills these needs and others.

### SUMMARY OF THE INVENTION

Briefly, and in general terms, the invention is directed to an EP catheter system and method for ablating biological tissue within a biological site and for determining the amount of RF power delivered to biological tissue during ablation.

In one aspect, the invention relates to a system for delivering RF energy to a load or ablation site, such as biological tissue. The system includes a catheter with an electrode that is adapted to be positioned at the load and a voltage-measurement reference device that is adapted to be positioned about the load such that the load is generally located between the electrode and the voltage-measurement reference device. A first wire and a second wire are each electrically connected to the electrode. The system further includes a power control system that delivers RF current to the load through the first wire and the electrode and measures the voltage across the load between the second wire and the voltage-measurement reference device.

By measuring the voltage across the load between the second wire and the voltage-measurement reference device the system measures the voltage at the load, as opposed to the source, as is done in known devices such as shown in FIG. 10. By measuring the power at the load, the voltage measurement, and thus the power reading, provided by the system avoids the measurement inaccuracies generally associated with transmission line losses or other inductively or capacitively reactive elements.

In a detailed aspect, the power control system measures the RF current through the first wire and determines the power delivered to the load using the measured RF current and voltage. In a further detailed aspect, the power control system compares the delivered power to a maximum power level and prevents the power from exceeding the maximum power level. In a still further detailed aspect, the power control system provides RF energy through a power output having an on/off duty cycle and the power control system prevents the power from exceeding the maximum power level by decreasing the duty cycle as the delivered power approaches the maximum power level.

In another facet, the invention relates to a system for delivering RF energy to biological tissue. The system includes a catheter having a plurality of ablation electrodes that are adapted to be positioned at the tissue. The system also includes a current-return electrode and a voltage-measurement reference device,each adapted to be positioned about the tissue such that the tissue is generally located between the plurality of ablation electrodes and each of the current-return electrode and the voltage-measurement reference device. At least one of a plurality of first wires and second wires are connected to at least one of the ablation electrodes. The system further includes a power control system that delivers RF current through the first wires and the ablation electrodes to the tissue between the ablation electrodes and the current-return electrode and measures the voltage across the tissue between the second wires and the voltage-measurement reference device.

In a detailed facet, the power control system measures the RF current through each of the first wires and, for each of the first wires, determines the power delivered to the tissue using the measured RF current and voltage. In another detailed facet, the first and second wires function as thermocouple leads which, in combination with the ablation electrode to which they are electrically connected, form a thermocouple and the power control system monitors the voltage across the thermocouple leads and determines the temperature at the ablation electrode. In further detailed facets, the power control system may be adapted to measure temperature during the delivery of RF current or alternatively, when RF current is not being delivered, such as during the off portion of a duty cycled power output.

In another aspect, the invention relates to a system for delivering RF energy to tissue generally located between an ablation electrode and a current-return electrode. The system includes means for delivering RF current through the tissue between the ablation electrode and the current-return electrode and means for measuring the voltage across the tissue between the ablation electrode and a voltage-measurement reference device.

In a detailed facet, the means for delivering RF current through the tissue includes a first wire electrically attached to the ablation electrode and a power control system that outputs RF current to the first wire and the ablation electrode and maintains the current-return electrode at a voltage level sufficient to establish a voltage difference between the ablation electrode and the current-return electrode. In another detailed facet, the means for measuring the voltage across the tissue includes a second wire electrically connected to the ablation electrode, a voltage-measurement return lead electrically connected to the voltage-measurement reference device and means for measuring the voltage between the second wire and the voltage-measurement return lead.

In another aspect, the invention relates to a method of delivering RF energy to tissue. The method includes delivering RF current through the tissue between an ablation electrode and a current-return electrode and measuring the voltage across the tissue between the ablation electrode and a voltage-measurement reference device.

In yet another aspect, the invention relates to a method of monitoring the power delivered to a tissue load during the delivery of RF energy to the load. The method includes measuring the RF current being delivered to the load, measuring the voltage at the load and determining the power using the measured RF current and measured voltage.

In a detailed facet of the method, measuring the voltage at the load includes positioning an electrode having a first wire and a second wire each electrically connected thereto, at the load. The method further includes positioning a voltage-measurement reference device about the load such that the load is generally located between the electrode and the voltage-measurement reference device and measuring the voltage across the second wire and the voltage-measurement reference device while delivering RF current to the load through the first wire. In another detailed facet of the method, measuring the RF current being delivered to the load includes positioning an electrode having a first wire electrically connected thereto at the load, delivering RF current to the load through the first wire and the electrode and measuring the RF current through the first wire.

In another aspect, the invention relates to a system for delivering RF energy to a load. The system includes an ablation catheter having an ablation electrode that is adapted to be positioned at the load. The system also includes a voltage-measurement electrode adapted to be positioned in proximity to the ablation electrode and a voltage-measurement reference device adapted to be positioned about the load such that the load is generally located between the ablation electrode and the voltage-measurement reference device. Also included in the system are a first wire that is electrically connected to the ablation electrode and a second wire that is electrically connected to the voltage-measurement electrode. The system further includes a power control system that is adapted to deliver RF current to the load through the first wire and the ablation electrode and to measure the voltage across the load between the second wire and the voltage-measurement reference device.

These and other aspects and advantages of the invention will become apparent from the following detailed description and the accompanying drawings which illustrate by way of example the features of the invention.

### BRIEF DESCRIPTION OF THE DRAWINGS

FIG. 1 is a schematic block diagram of an RF ablation system configured in accordance with aspects of the invention including a power control system (“PCS”), two current-return electrodes, one voltage-measurement reference device and a catheter system;

FIG. 2 is a diagram of the catheter system of FIG. 1 presenting more detail that includes a handle and a catheter shaft having a preformed distal segment carrying an electrode system;

FIG. 3 is a detailed schematic diagram of one configuration of the electrode system of FIG. 2 having a tip electrode and several band electrodes arranged in a linear array with various drive wires and temperature leads attached;

FIG. 4a is a diagram of another configuration of the electrode system of FIG. 2 having a tip electrode and several band electrodes arranged in a circular loop;

FIG. 4b is a diagram of another configuration of the electrode system of FIG. 2 having a central electrode and four orthogonally arranged branch electrodes;

FIG. 5 is a cross-sectional view of the distal segment of FIG. 3 taken along line 5-5, depicting the attachment points of a drive wire and two temperature leads;

FIG. 6 is a cross-sectional view of the distal segment of FIG. 3 taken along line 6-6, depicting the attachment points of a drive wire and one temperature lead;

FIGS. 7A and 7B form a block diagram presenting a detailed configuration of one embodiment of the RF ablation system of FIG. 1 as it relates to an electrode having a drive wire and two temperature leads as shown in FIGS. 3 and 5;

FIGS. 8A and 8B form a block diagram of a multi-channel ablation system configured in accordance with the configuration of FIGS. 7A and 7B wherein a single PCS microprocessor controls the application of ablation energy to each channel individually;

FIG. 9 is a block diagram of a multi-channel ablation system depicting circuitry for monitoring the power delivered to a tissue load; and

FIG. 10 is a block diagram of a prior art system for monitoring the power delivered to a tissue load.

### DETAILED DESCRIPTION OF THE PREFERRED EMBODIMENTS

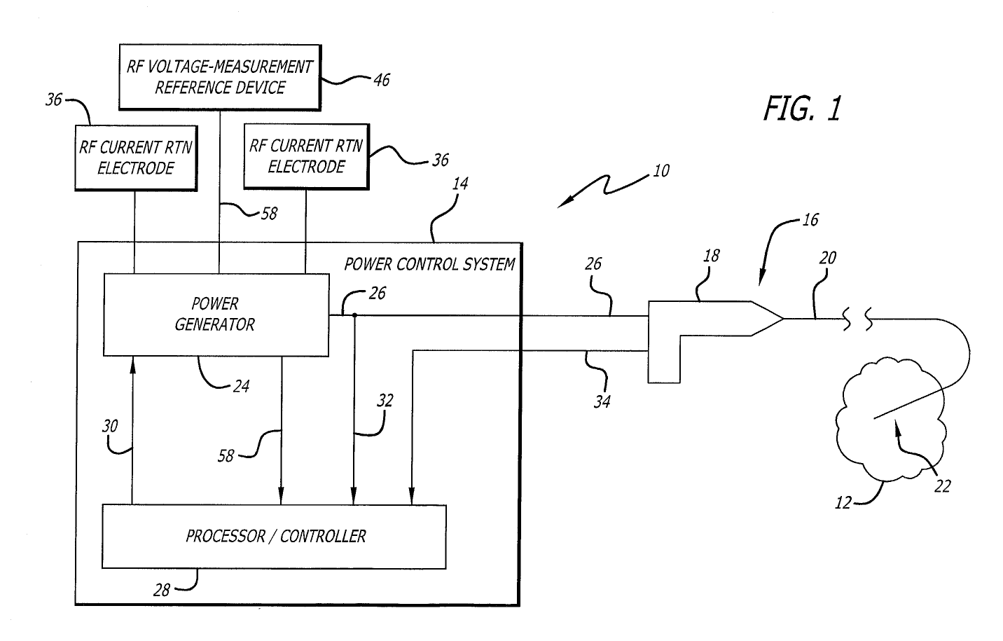

Turning now to the drawings, in which like reference numerals are used to designate like or corresponding elements among the several figures, in FIG. 1 there is shown a system 10 for use in RF ablation therapy of a biological site 12, e.g., the atrium or ventricle of the heart. The system 10 includes an RF power control system 14 and a catheter system 16. The catheter system 16 includes a handle 18 and a steerable catheter shaft 20 having a distal segment 22. The distal segment 22 carries an electrode system (not shown) and is capable of being percutaneously introduced into a biological site 12.

The power control system 14 includes an RF power generator 24, through which it provides RF power 26 to the catheter system 16. Although the power 26 provided by the power generator 24 is illustrated as a single output, the power generator may have any number of channels through which it provides a plurality of power outputs, each characterized by a waveform having an associated amplitude, frequency, phase and duty cycle having alternating instances of peak power, i.e., “on” periods, and very low or zero power, i.e., “off” periods.

The system 10 also includes one or more RF current-return electrodes 36 or backplates. The current-return electrodes 36 are connected to the power generator 24 and generally provides a return path for the power 26 delivered to the biological site 12 through the catheter system 16. The system further includes an RF voltage-measurement reference device 46, such as a backplate, that is connected to the power generator 24. The RF voltage at the voltage-measurement reference device 46 is monitored by the processor/controller 28 over a voltage-measurement return lead 58. Current-return electrodes 36 and the voltage-measurement reference device 46 are currently available as self adhesive pads with an electrically conductive gel region and are typically affixed to an exterior surface of the biological subject.

The operation of the power generator 24 is controlled by a processor/controller 28 which outputs control signals 30 to the power generator. The processor/controller 28 monitors the RF current provided by the power generator 24 over a current monitor line 32. In addition, the processor/controller 28 also monitors one or more voltages at the catheter system 16 over voltage monitor lines 34 and the voltage-measurement return lead 58. Only one voltage monitor line 34 is shown in FIG. 1, although more may be present. As explained further below, the monitored voltages include both RF voltages and thermocouple (TC) voltages. The TC voltages are used by the processor/controller 28 to determine the temperature at the electrode system while the RF voltages are used to, in conjunction with the monitored RF current, determine the RF power being provided to the electrode system. Based on the power and the temperature determinations, the processor/controller 28 adjusts the operation of the power generator 24.

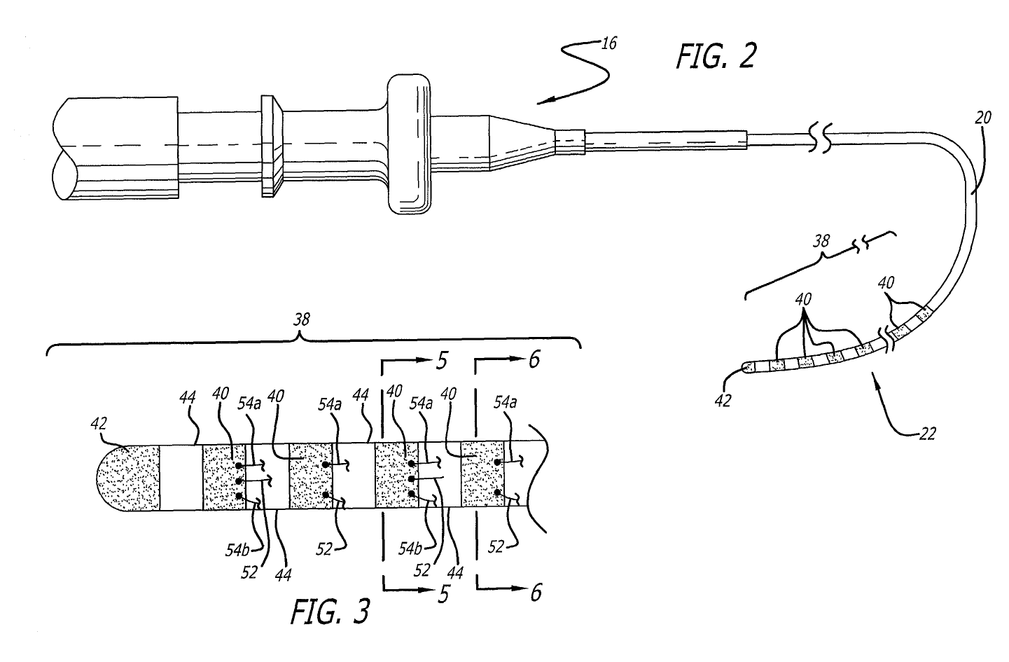

As shown in FIGS. 2 and 3, the distal segment 22 of the catheter system 16 includes an electrode system 38. In FIG. 3, the electrode system 38 is shown in schematic form with the components drawn in more detail to more clearly illustrate the relationship between the components. Preferred embodiments of the electrode system 38 includes six or twelve band electrodes 40 arranged in a substantially linear array along the distal segment 22 of the catheter shaft 20, although any number of electrodes may be used. The electrode system 38 may include a tip electrode 42. (For clarity of illustration, only six band electrodes 40 are shown in FIG. 2 and only four band electrodes 40 are shown in FIG. 3 although as stated, a preferred embodiment may include many more.) The band electrodes 40 are arranged so that there is an electrically non-conductive space 44 between adjacent electrodes. The electrodes 40 are spaced close enough to each other such that a continuous lesion is formed between adjacent electrodes by the bipolar current flowing between the electrodes. In one configuration of the electrode system 38, the width of the band electrodes 40 is 3 mm and the space 44 between the electrodes is 4 mm. The total length of the electrode system 38, as such, is approximately 8 cm for twelve band electrodes.

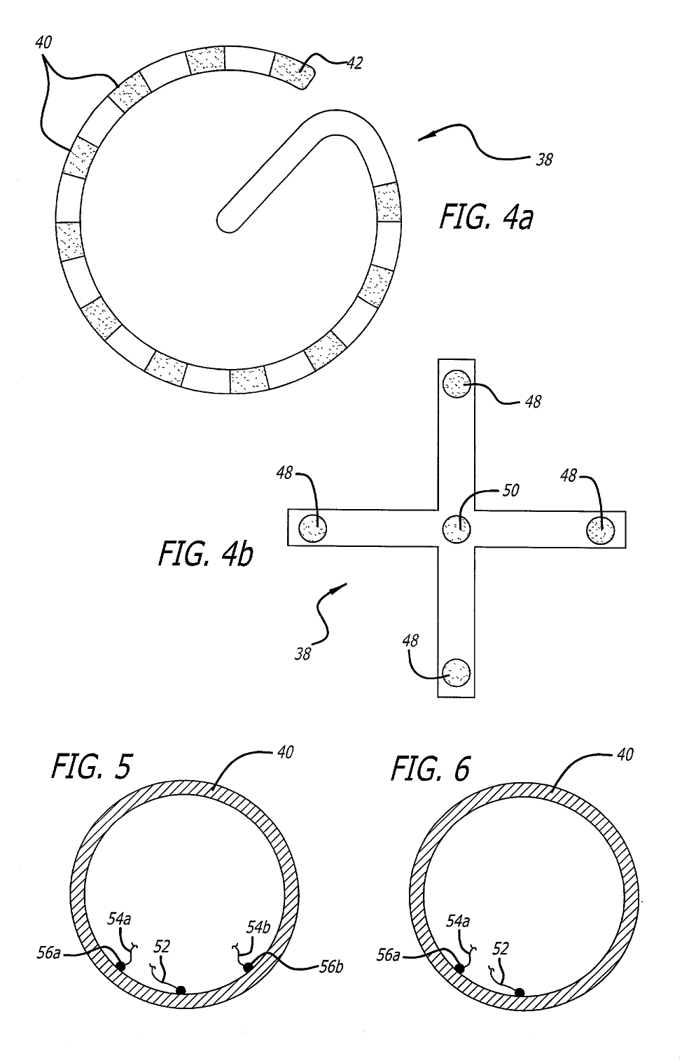

The arrangement of the electrodes 40, 42 is not limited to a linear array and may take the form of curvilinear arrays or other patterns. For example, as shown in FIG. 4a, the tip electrode 42 and the band electrodes 40 may be arranged in a circular loop. Alternatively, as shown in FIG. 4b, the electrode system 38 may include several branch electrodes 48 orthogonally arranged around a central electrode 50, such as that disclosed in U.S. Pat. No. 5,383,917. A substantially linear or curvilinear array is preferred for certain therapeutic procedures, such as treatment of atrial fibrillation, in which linear lesions of typically 4 to 8 cm in length are desired.

The band electrodes 40 and tip electrode 42 are formed of a material having a significantly higher thermal conductivity than that of the biological tissue to be ablated. Possible materials include silver, gold, chromium, aluminum, molybdenum, tungsten, nickel, platinum, and platinum/10% iridium. Because of the difference in thermal conductivity between the electrodes 40, 42 and the tissue, the electrodes cool off more rapidly in the flowing fluids at the biological site. The band electrodes 40 are sized so that the surface area available for contact with fluid in the heart, e.g., blood, is sufficient to allow for efficient heat dissipation from the electrodes to the surrounding blood. In a preferred embodiment, the electrodes 40 are 7 French (2.3 mm in diameter) with a length of 3 mm and a thickness in the range of about 0.002 mm to about 0.020 mm.

With reference to FIG. 3, in one configuration of the electrode system 38, each of the band electrodes 40 has an RF current-carrying drive wire 52 and either one or two temperature leads 54a, 54b electrically attached. In other embodiments any number of temperature leads may be used. In the embodiment depicted, alternate electrodes 40 have two temperature leads 54a, 54b, with the remaining electrodes having only one temperature lead 54a. The drive wires 52 provide RF power to each electrode for ablation purposes while the temperature leads 54a, 54b allow for monitoring of the temperature of the electrode system 38 at various points along its length.

As shown in FIG. 5, for band electrodes 40 having two temperature leads 54a, 54b, the leads are attached to the inside surface of the band electrode 40 approximately 60 degrees apart along the circumference of the electrode. Each of the temperature leads 54a, 54b, in combination with the drive wire 52 and the band electrode 40, form a thermocouple 56a, 56b such as described in U.S. Pat. No. 6,042,580, the disclosure of which is hereby incorporated by reference. In a preferred embodiment, the drive wires 52 are formed of alloy 11 while the temperature leads 54a, 54b are formed of constantan. For band electrodes 40 with only one temperature lead 54a, as shown in FIG. 6, only one thermocouple 56a is formed. In other configurations, some electrodes may have more than two temperature leads attached while some may have no temperature leads attached. Accordingly, various numbers of temperature leads may be attached to the electrodes in different combinations to form any number of thermocouples.

With reference to FIGS. 1 and 5, the TC voltage potential established across the drive wire 52 and each of the temperature leads 54a, 54b may be periodically monitored by the processor/controller 28 over the TC/RF voltage monitor line 34. In this sense, the TC/RF voltage monitor line 34 includes three lines, one for each of the three leads 52, 54a, 54b. The TC voltage potential across the drive wire 52 and the first temperature lead 54a is indicative of the temperature at the attachment point of the first temperature lead to the electrode 40. Likewise, the TC voltage potential across the drive wire 52 and the second temperature lead 54b is indicative of the temperature at the attachment point of the second temperature lead to the electrode 40. As explained further below, these TC voltage potentials are used to determine the temperature at the electrode-tissue interface.

With continued reference to FIGS. 1 and 5, the voltage potential between the voltage-measurement reference device 46 and each of the electrodes 40 may be periodically monitored by the processor/controller 28 over the TC/RF voltage monitor line 34, through temperature leads 54a, 54b, and the voltage-measurement return lead 58. As explained further below, this RF voltage potential is used to determine the power being delivered to the biological tissue.

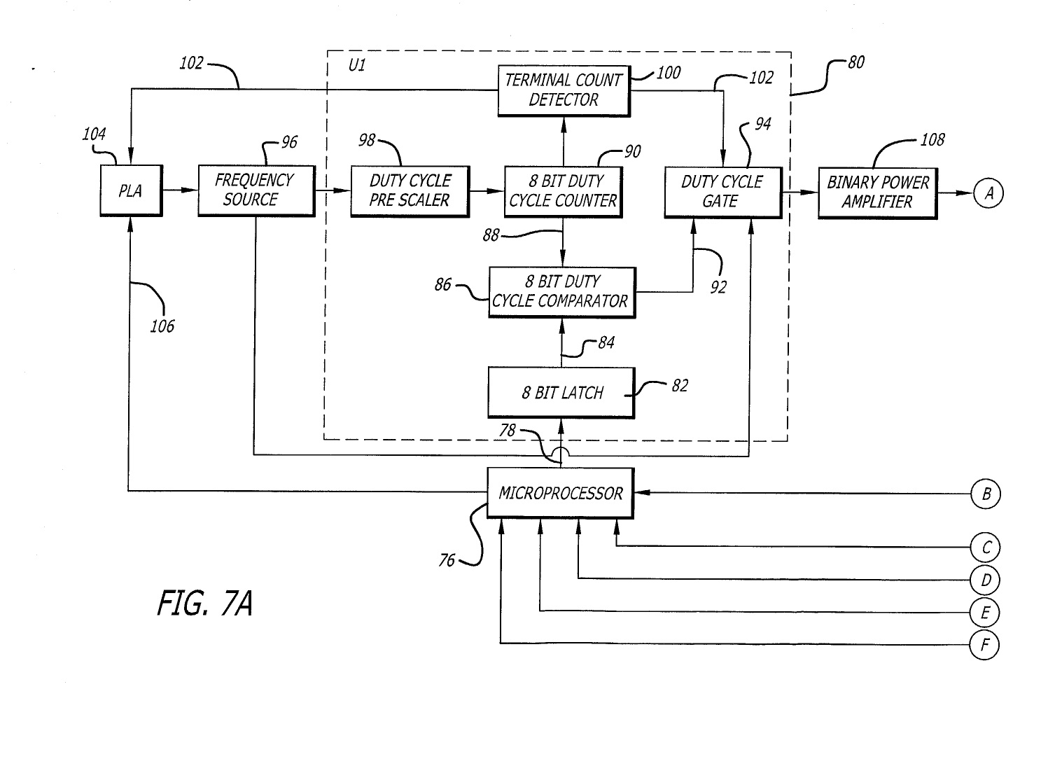

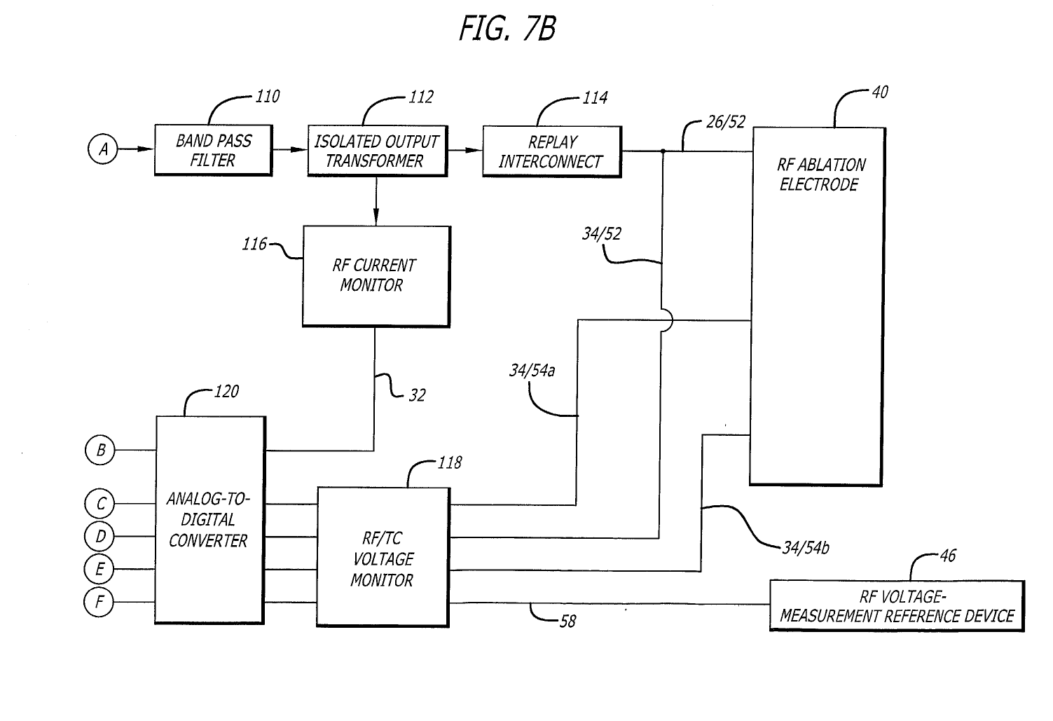

With reference to FIGS. 7A and 7B, there is shown a block diagram of an ablation system which incorporates aspects of the invention. In FIG. 7A, a microprocessor 76, which is part of the processor/controller 28 (FIG. 1), provides a duty cycle control signal 78 to a duty cycle generator (“DCG”) 80. In this case, the duty cycle generator 80 receives the control signal 78 by an 8-bit latch 82. The latch 82 provides an 8-bit signal 84 to a duty cycle comparator 86. The comparator 86 compares the 8-bit signal 84 to a count 88 from an 8-bit duty cycle counter 90 and if the count is the same, provides a duty cycle off signal 92 to the duty cycle gate 94. The gate 94 is connected to a frequency source (“FS”) 96, such as an oscillator that produces an approximate 500 kHz signal. When the gate 94 receives the duty cycle off signal 92 from the comparator 86, it stops its output of the frequency source signal through the gate and no output exists.

At a frequency of approximately 500 kHz, an 8-bit control has a period or time frame of 0.5 msec. At a fifty-percent duty cycle, the electrode is in the off period only 0.25 msec. The period or time frame is lengthened by use of a prescalar 98 interposed between the frequency source 96 and the counter 90. In one embodiment, the prescalar 98 lengthens the period to 4 msec thus allowing for a 2 msec off period during a fifty-percent duty cycle. Other lengths of the period may be used depending on the circumstances. It has been found that a ten percent duty cycle is particularly effective in ablating heart tissue.

A terminal count detector 100 detects the last count of the period and sends a terminal count signal 102 to the gate 94 which resets the gate for continued output of the frequency source signal. This then begins the on period of the duty cycle and the counter 90 begins its count again. In one preferred embodiment, the duty cycle is set at fifty percent and the 8-bit latch is accordingly set to 128. In another embodiment, the duty cycle is set at ten percent.

A programmable logic array (“PLA”) 104 receives phase control signals 106 from the microprocessor 76 and controls the phase of the frequency source 96 accordingly. In one embodiment, the PLA 104 receives the terminal count signal 102 from the terminal count detector 100 and only permits phase changes after receiving that terminal count signal.

The output signal from the gate 94 during the on-period of the duty cycle is provided to a RF binary power amplifier (“BPA”) 108 that chops a 24 volt DC source. The chopped signals are then filtered with a band pass filter (“BPF”) 110 to convert the somewhat square wave to a sine wave. The band pass filter 110 in one embodiment is centered at approximately 500 kHz. The filtered signal is then provided to the primary sided of an isolated output transformer (“IOT”) 112 that amplifies the signal to a much higher level, for example 350 volts peak-to-peak. This signal is then sent to a relay interconnect (“RI”) 114 before it is provided as a RF power output OUTn 26 to the electrode 40.

RF current measurement circuitry (“CM”) 116 provides a RF current measurement to the microprocessor 76 over the RF current monitor line 32. The RF current measurement is actually a voltage measurement that is representative of the current passing through a resistor of known value within the RF current measurement circuitry 116. The current measurement is indicative of the RF current of the power output 26 provided to the electrode over the drive wire 52. RF/TC voltage-measurement circuitry (“VM”) 118 provides various voltage measurements including a measurement of the RF voltage at the load, i.e., the tissue between the electrode 40 and the voltage-measurement reference device 46, by measuring the RF voltage across either of the temperature leads 54a, 54b and the voltage-measurement return lead 58. The RF/TC voltage-measurement circuitry 118 also provides a measurement of the TC voltage potential across the drive wire 52 and each of the temperature leads 54a, 54b. These TC voltage potentials are indicative of the temperature at the electrode 40. In each instance, the RF current and RF/TC voltage measurements are converted to digital form by an analog-to-digital converter (“ADC”) 120 prior to processing. Although the RF/TC voltage-measurement circuitry 118 is depicted as a single block, separate circuitry may be used to provide the various measurements.

The power control system 14 may be conceptually described as having two states of operation, a RF power application state and a temperature measurement state. During a temperature measurement state, the microprocessor 76 monitors the TC voltage potential across the first temperature lead 54a and the drive wire 52 and the second temperature lead 54b and the drive wire based on the TC voltage measurements from the RF/TC voltage-measurement circuitry 118, and determines the temperatures at the electrode based on these voltages. During a RF power application state, the microprocessor 76 receives the RF current measurement from the RF current measurement circuitry 116 and monitors the RF voltage potential across one of the temperature leads 54a, 54b and the RF voltage-measurement return lead 58 through the RF/TC voltage-measurement circuitry 118. With these RF current and RF voltage measurements, the microprocessor 76 determines the RF power being delivered to the tissue.

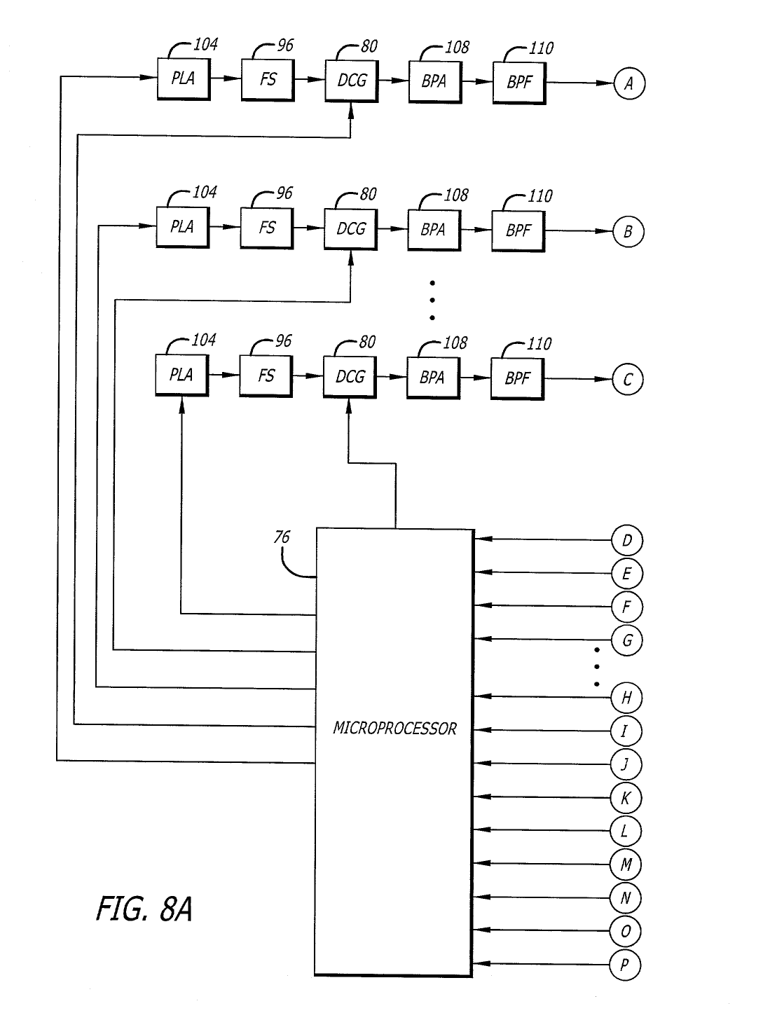

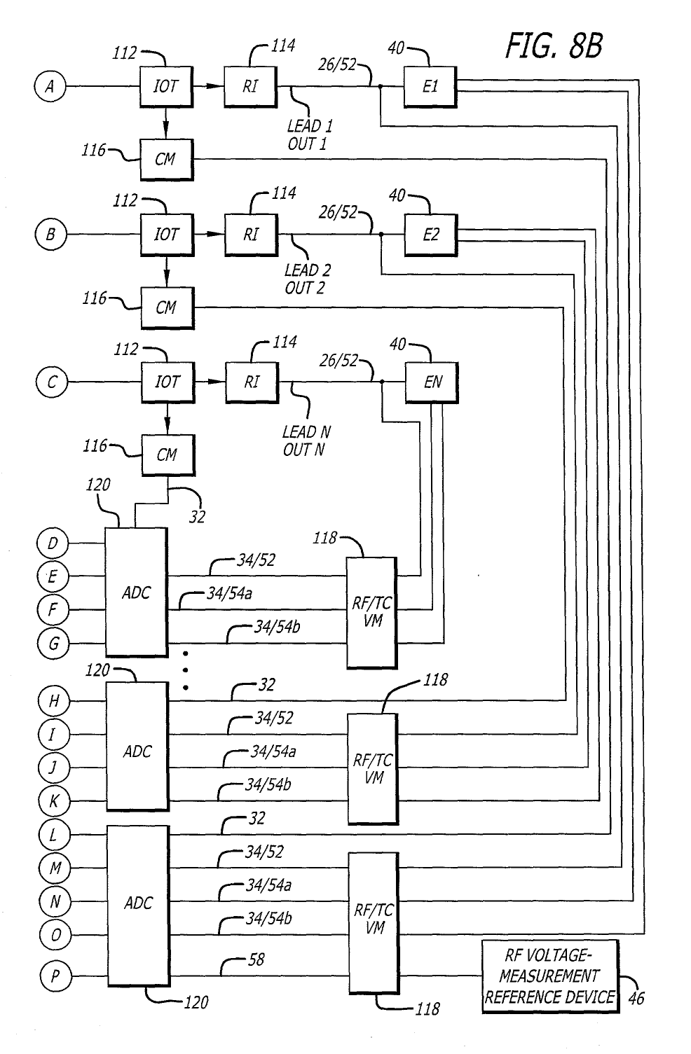

Referring now to FIGS. 8A and 8B, a block diagram of a multi-channel ablation system for use with a catheter system having a plurality of electrodes 40 is shown. Although only three complete channels are shown, the system comprises many more as indicated by the successive dots. Those channels are not shown in FIGS. 8A and 8B to preserve clarity of illustration. The single microprocessor 76, which again is part of the processor/controller 28 (FIG. 1), controls the duty cycle and the phase of each channel individually in this embodiment. Each channel shown comprises the same elements and each channel produces its own RF power output 26 (OUT1, OUT2, through OUTn where “n” is the total number of channels) on a respective RF current-carrying drive wire 52 (WIRE 1, WIRE 2, through WIRE n where “n” is the total number of leads) to an electrode.

With respect to each electrode 40, the microprocessor 76 receives RF current and RF/TC voltage measurements from respective RF current measurement and RF/TC voltage-measurement circuitry 116, 118. Using the voltage potentials and current measurements provided by the various lines, the microprocessor 76 determines the temperature at each electrode 40 and the RF power delivered across the tissue through each electrode, as previously described with respect to FIGS. 7A and 7B.

With reference to FIG. 9, in operation, the current-return electrodes 36 and the voltage-measurement reference device 46 are positioned relative to the ablation electrodes 40 such that the tissue load 62 is positioned between the electrode and the reference device. The current-return electrodes 36 and the voltage-measurement reference device 46 are depicted on top of each other for ease in illustrating system operation. In practice, each of the electrode 36 and the reference device 46 contact the tissue load 62. In a typical operation, the ablation electrodes are positioned within a body cavity such as the atrium, adjacent to the tissue 62 to be ablated. The current-return electrodes 36 and voltage-measurement reference device 46 are usually positioned exterior the body cavity about the tissue 62 such that the load is positioned between the ablation electrodes and the current-return electrodes 36 and the voltage-measurement reference device 46.

The power control system 14 provides RF power outputs 26 to one or more of the electrodes 40 through one or more output channels 60 shown schematically as RF amplifiers. The power outputs 26 are provided such that bipolar current flows between adjacent electrodes, unipolar current flows between electrodes and a current-return electrode 36 or a combination of both. In one embodiment the power outputs 26 are offset in phase to establish a voltage potential between the electrodes 40, as described in U.S. Pat. No. 6,050,994, the disclosure of which is hereby incorporated by reference. During operation, the power control system 14 monitors the temperature at each electrode 40 and the amount of power being delivered across the tissue load 62 through each electrode.

In accordance with the temperature monitoring feature of the power control system, the temperature measurements provided by the thermocouples 56a, 56b (FIGS. 5 and 6) are used by the processor/controller 28 (FIG. 1) to monitor the electrodes 40 for unacceptable temperature conditions. Such conditions are described in detail in U.S. application Ser. No. 09/738,032, the disclosure of which is hereby incorporated by reference. For example, in one configuration of the system, if the measured temperature at the interface between the tissue and an electrode 40 is between 5° C. and 9° C. greater than a target temperature programmed in the processor/controller 28, a control signal 32 is sent to the power generator 24 to reduce the duty cycle of the power output 26 being sent to the particular electrode to allow the electrode-tissue interface temperature to cool off. Once the interface is cooled off, the processor/controller 28, may if necessary, incrementally increases the duty cycle of the power output 26, thereby increasing the power to the electrode 40 until the electrode-tissue interface temperature settles to a temperature near the target temperature.

In general, the processor/controller 28 is programmed to control the RF power such that the closer the electrode-tissue interface temperature is to the target temperature the lesser the rate of change of the duty cycle of the power output 28. For example, if the measured temperature is 20° C. less than the target temperature, the duty cycle may be set relatively high in order to increase the electrode-tissue interface temperature rapidly. As the measured temperature increases and the difference between it and the target temperature becomes smaller, the duty cycle may be reduced in order to settle in on the target temperature and to avoid exceeding the target temperature by a predetermined amount.

As previously mentioned, in addition to the temperature monitoring feature, the power control system also includes a RF power monitoring feature. While the temperature monitoring feature protects against the formation of coagulum by preventing the overheating of the tissue surface and the electrodes, the power monitoring feature protects against tissue dessication by preventing the delivery of excessive RF power to the tissue. The RF power monitoring feature operates independent of the temperature monitoring feature and provides an additional level of protection in situations where the protection provided by the temperature monitoring feature may be inadequate. For example, when ablating around an area of high blood flow, such as the pulmonary vein, the temperatures at the tissue surface and the electrodes may experience a cooling effect due to the blood flow which may in turn lead to inaccurate temperature readings. Thus, RF power of a greater level than necessary may continue to be provided to the tissue. In the case of the pulmonary vein, this excessive delivery of RF power may result in stenosis.

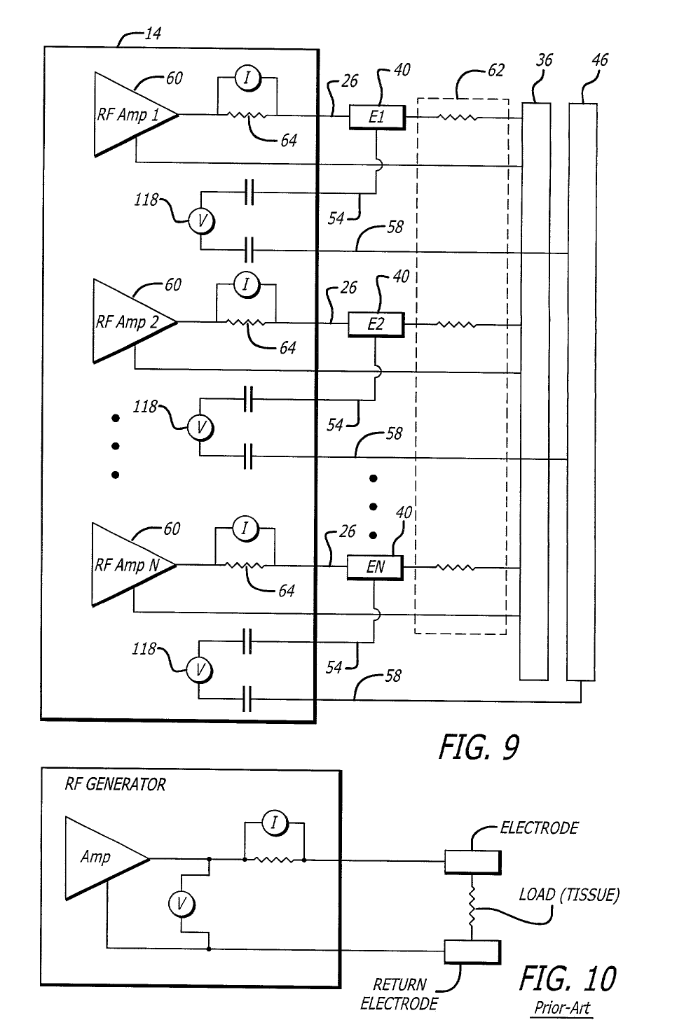

With reference to FIG. 9, RF power is monitored by determining the amount of RF current I being delivered to the electrode 40 and the RF voltage V across the tissue load 62 between each of the electrodes 40 and the voltage-measurement reference device 46. The current is measured by the previously described RF current measurement circuitry 116 (FIG. 7B) at the output of the output channels 60 (FIG. 9) across a current sense resistor 64 that is in series with the power output 26 wire and the load 62. The RF voltage across the load 62 is measured at the load using the electrodes 40, the voltage-measurement reference device 46 and the previously described RF/TC voltage-measurement circuitry 118 (FIG. 7B). In this regard, it is significant to note that the RF voltage measured by the power control system 14 is measured at the load 62 as opposed to the source, as is done in known devices such as shown in FIG. 10. By measuring the RF voltage at the load 62, the voltage reading provided by the system 14 avoids the measurement inaccuracies generally associated with transmission line losses or other inductively or capacitively reactive elements. This more accurate RF voltage reading, in turn, provides a more accurate RF power reading.

With continued reference to FIG. 9, in order to provide an accurate RF voltage measurement, the RF voltage-measurement return lead 58 and temperature leads 54 carry near zero current. This is accomplished by configuring the RF/TC voltage-measurement circuitry 118 to have high impedance inputs that draw near-zero current through the leads 58, 54. In addition, to isolate the tissue load 62 from potentially harmful DC voltages, the electrode 40 and the voltage-measurement reference device 46 are capacitively coupled to the RF/TC voltage-measurement circuitry 118. Furthermore, the RF/TC voltage-measurement circuitry 118 is isolated from ground and in one configuration is at 5 kV DC.

The power control system 14 monitors the RF power being delivered to the tissue load 62 and protects against excessive power delivery accordingly. In one configuration, a maximum level of RF power delivery, e.g., 25-30 watts, is programmed into the processor/controller 28 by front panel controls. For each electrode 40, the power control system 14 periodically or continuously measures the RF power at the load 62 and adjusts the power output 26 to that electrode such that the power does not exceed the maximum level. In one configuration of the power control system 14, the level of RF energy is adjusted by controlling the duty cycle of the power output 26. In alternate configurations of the power control system 14, adjustments to energy outputs may be made by adjusting the amplitude of the power output 26.

As previously described, in addition to monitoring the RF power delivery, the power control system may also monitor the temperature at the electrodes 40. Temperature monitoring may occur separate from, or simultaneously with, power monitoring. In one embodiment, when the RF power output 26 is characterized by a duty cycle, temperature and power monitoring may occur separately with the temperature monitoring taking place during the off portion of the duty cycle and the power monitoring taking place during the on portion of the duty cycle. Monitoring temperatures during the off portion prevents any RF signal noise present in the drive wire 52 from interfering with the temperature measurement. Alternatively, the temperature measurements may be taken during the application of RF energy, in which case the RF/TC voltage-measurement circuitry 118 includes filter circuitry that filters out the RF noise present in the drive wire 52. In this configuration, the microprocessor 76 (FIG. 8) simultaneously monitors the voltage potentials between the temperature leads 54a, 54b and the drive wire 52 and between one of the temperature leads 54a, 54b and the voltage-measurement return lead 58. The former provides a temperature related measurement while the latter provides a RF power related measurement.

Both the drive wire 52 and the temperature leads 54a, 54b may be described as having dual functions. With respect to the drive wire 52, it functions as both a RF current delivery wire and as a thermocouple lead. Each of the temperature leads 54a, 54b functions as a lead for measuring RF voltage at the load and as a thermocouple lead.

While the various embodiments of the invention have been described as using thermocouple temperature leads to provide a measurement of voltage at the load, alternate configurations of the system may employ other types of thermal sensors such as thermistors, resistance temperature detectors (RTD) and fluoroptic probes, which do not have leads capable of providing dual functions. In such cases, the electrode system may have a dedicated lead for providing voltage measurements and the load.

Also, while the voltage-measurement reference device 46 has been described as a pad type electrode located on the exterior of a patient, in alternative configurations an electrode carried by a catheter may function as a voltage-measurement reference device. In this type of configuration, the voltage-measurement reference device may be carried by a catheter other than the ablation catheter and may be introduced into a portion of a body, e.g., the right ventricle, while the ablation electrodes are introduced into another portion of the body, e.g., the right atrium, with the target tissue located in between.

In other configurations, the voltage-measurement reference device 46 may be carried by the ablation catheter. For example, with reference to FIG. 3, the ablation catheter may include a plurality of ablation electrodes 40, each with at least one temperature lead 54a, 54b. The processor/controller 28 may be programmed to use any one of the ablation electrodes 40 as a voltage-measurement reference device 46 and monitor the RF voltage potential between the reference device 46 and another one of the ablation electrodes 40, preferably an adjacent electrode. The RF voltage potential is measured across the temperature leads 54a, 54b of the reference device 46 and the electrode 40 to determine the RF voltage. In another configuration, the ablation catheter may include an additional electrode that functions exclusively as a voltage-measurement reference device 46.

In other arrangements, the voltage-measurement reference device 46 remains the backplate while the point of RF voltage measurement changes. In embodiments thus described the RF voltage is measured at the point where RF energy is applied, i.e., at the ablation electrode 40. The point of measurement could be at other sites, such as an independent electrode on the ablation catheter whose sole function would be to measure RF voltage. Alternatively, the point of measurement could be an electrode on a separate catheter placed in close proximity to the ablation catheter during an ablation procedure.

It will be apparent from the foregoing that while particular forms of the invention have been illustrated and described, various modifications can be made without departing from the spirit and scope of the invention. Accordingly, it is not intended that the invention be limited, except as by the appended claims.

What is claimed is:

1. A system for delivering RF energy to a load, said system comprising:
a catheter having an electrode adapted to be positioned at the load;
a voltage-measurement reference device adapted to be positioned about the load such that the load is generally located between the electrode and the voltage-measurement reference device;
a first wire and a second wire each electrically connected to the electrode; and
a power control system adapted to deliver RF current to the load through the first wire and the electrode and to measure the voltage across the load between the second wire and the voltage-measurement reference device.

2. The system of claim 1 wherein the power control system is adapted to:
measure the RF current through the first wire; and
determine the power delivered to the load using the measured RF current and voltage.

3. The system of claim 2 wherein the power control system is adapted to:
compare the delivered power to a maximum power level; and
prevent the power from exceeding the maximum power level.

4. The system of claim 3 wherein the power control system provides RF energy through a power output having an on/off duty cycle and the power control system is adapted to prevent the power from exceeding the maximum power level by decreasing the duty cycle as the delivered power approaches the maximum power level.

5. The system of claim 1 wherein the power control system provides RF energy through a power output having an on/off duty cycle and the power control system is adapted to measure the voltage across the load during the on portion of the duty cycle.

6. The system of claim 1 wherein the first and second wires function as thermocouple leads which, in combination with the electrode to which they are electrically connected, form a thermocouple and the power control system is adapted to measure the voltage across the thermocouple leads and determine the temperature at the electrode.

7. The system of claim 6 wherein the power control system is adapted to measure temperature during the delivery of RF current.

8. The system of claim 6 wherein the power control system is adapted to measure temperature when RF current is not being delivered.

9. The system of claim 6 wherein the power control system provides RF energy through a power output having an on/off duty cycle and the power control system is adapted to measure the temperature at the electrode during the off portion of the duty cycle.

10. The system of claim 1 wherein the voltage-measurement reference device comprises:
a backplate; and
a voltage-measurement return lead having a first end electrically connected to the backplate and a second end in electrical communication with the power control system.

11. The system of claim 1 wherein the voltage-measurement reference device comprises:
an electrode; and
a voltage-measurement return lead having a first end electrically connected to the electrode and a second end in electrical communication with the power control system.

12. The system of claim 11 wherein the electrode is carried by the catheter.

13. The system of claim 11 wherein the electrode is carried by a second catheter.

14. A system for delivering RF energy to biological tissue, said system comprising:
a catheter having a plurality of ablation electrodes adapted to be positioned at the tissue;
a current-return electrode and a voltage-measurement reference device each adapted to be positioned about the tissue such that the tissue is generally located between the plurality of ablation electrodes and each of the current-return electrode and the voltage-measurement reference device;
a plurality of first wires and second wires, at least one of each electrically connected to at least one of the ablation electrodes; and
a power control system adapted to deliver RF current through the first wires and ablation electrodes to the tissue between the ablation electrodes and the current-return electrode and to measure the voltage across the tissue between the second wires and the voltage-measurement reference device.

15. The system of claim 14 wherein the power control system is adapted to:
measure the RF current through each of the first wires; and
for each of the first wires, determine the power delivered to the tissue using the measured RF current and voltage.

16. The system of claim 14 wherein the first and second wires function as thermocouple leads that, in combination with the ablation electrode to which they are electrically attached, form a thermocouple and the power control system is adapted to monitor the voltage across the thermocouple leads and determine the temperature at the ablation electrode.

17. A system for delivering RF energy to tissue generally located between an ablation electrode and a current-return electrode, said system comprising:
means for delivering RF current through the tissue between the ablation electrode and the current-return electrode; and
means for measuring the RF voltage across the tissue between the ablation electrode and a voltage-measurement reference device.

18. The system of claim 17 wherein the means for delivering RF current through the tissue comprises:
a first wire electrically attached to the ablation electrode; and
a power control system adapted to output RF current to the first wire and the ablation electrode and maintain the current-return electrode at a voltage level sufficient to establish a voltage difference between the ablation electrode and the current-return electrode.

19. The system of claim 17 wherein the means for measuring the RF voltage across the tissue comprises:
a second wire electrically connected to the ablation electrode;
a voltage-measurement return lead electrically connected to the voltage-measurement reference device;
means for measuring the RF voltage between the second wire and the voltage-measurement return lead.

20. The system of claim 19 wherein the voltage-measurement reference device comprises a backplate.

21. The system of claim 19 wherein the voltage-measurement reference device comprises an electrode.

22. The system of claim 17 wherein the means for measuring the RF voltage across the tissue comprises:
a second wire electrically connected to an electrode positioned in proximity to the ablation electrode;
a voltage-measurement return lead electrically connected to the voltage-measurement reference device;
means for measuring the RF voltage between the second wire and the voltage-measurement return lead.

23. The system of claim 17 further comprising means for measuring the RF current through the first wire.

24. The system of claim 23 further comprising means for determining the power delivered to the tissue through the ablation electrode using the measured RF current and RF voltage.

25. A method of delivering RF energy to tissue comprising:
delivering RF current through the tissue between an ablation electrode and a current-return electrode; and
measuring the voltage across the tissue between the ablation electrode and a voltage-measurement reference device.

26. The method of claim 25 wherein delivering RF current through the tissue comprises delivering RF current through a first wire electrically attached to the ablation electrode while maintaining the current-return electrode at a voltage level sufficient to establish a voltage difference between the ablation electrode and the current-return electrode.

27. The method of claim 25 wherein measuring the voltage across the tissue comprises measuring the voltage difference across a second wire electrically connected to the ablation electrode and a voltage-measurement return lead electrically connected to the voltage-measurement reference device.

28. The method of claim 25 wherein measuring the voltage across the tissue comprises measuring the voltage difference across a second wire electrically connected to an electrode positioned in proximity to the ablation electrode and a voltage-measurement return lead electrically connected to the voltage-measurement reference device.

29. The method of claim 25 further comprising measuring the RF current through the first wire.

30. The method of claim 29 further comprising determining the power delivered to the tissue through the ablation electrode using the measured RF current and voltage.

31. A method of monitoring the power delivered to a tissue load during the delivery of RF energy to the load, said method comprising:
measuring the RF current being delivered to the load;
measuring the voltage at the load; and
determining the power using the measured RF current and measured voltage.

32. The method of claim 31 wherein measuring the voltage at the load comprises:
positioning an electrode at the load, the electrode having a first wire and a second wire each electrically connected thereto;
positioning a voltage-measurement reference device about the load such that the load is generally located between the electrode and the voltage-measurement reference device; and
measuring the voltage across the second wire and the voltage-measurement reference device while delivering RF current to the load through the first wire.

33. The method of claim 31 wherein measuring the RF current being delivered to the load comprises:
positioning an electrode at the load, the electrode having a first wire electrically connected thereto;
delivering RF current to the load through the first wire and the electrode; and
measuring the RF current through the first wire.

34. The method of claim 31 further comprising:
comparing the delivered power to a maximum power level; and
preventing the power from exceeding the maximum power level.

35. The method of claim 34 wherein the RF energy is delivered through a power output having an on/off duty cycle and preventing the power from exceeding the maximum power level comprises decreasing the duty cycle as the delivered power approaches the maximum power level.

36. The method of claim 34 wherein the RF energy is delivered through a power output having an amplitude and preventing the power from exceeding the maximum power level comprises decreasing the amplitude as the delivered power approaches the maximum power level.

37. The method of claim 31 wherein the RF energy is delivered through a power output having an on/off duty cycle and measuring the voltage at the load occurs during the on portion of the duty cycle.

38. The method of claim 31 further comprising measuring the temperature at the tissue load.

39. The method of claim 38 wherein measuring the temperature at the load comprises:
positioning an electrode at the load, the electrode having a first wire and a second wire each electrically connected thereto, wherein the first and second wires function as thermocouple leads which, in conjunction with the electrode to which they are connected, form a thermocouple; and
measuring the voltage across the first and second wires.

40. The method of claim 39 wherein the RF energy is provided through a power output having an on/off duty cycle and measuring the voltage across the first and second wires occurs during the off portion of the duty cycle.

41. A system for delivering RF energy to a load, said system comprising:
an ablation catheter having an ablation electrode adapted to be positioned at the load;
a voltage-measurement electrode adapted to be positioned in proximity to the ablation electrode;
a voltage-measurement reference device adapted to be positioned about the load such that the load is generally located between the ablation electrode and the voltage-measurement reference device;
a first wire electrically connected to the ablation electrode;
a second wire electrically connected to the voltage-measurement electrode; and
a power control system adapted to deliver RF current to the load through the first wire and the ablation electrode and to measure the voltage across the load between the second wire and the voltage-measurement reference device.

42. The system of claim 41 wherein the voltage-measurement electrode is carried by a catheter.

43. The system of claim 42 wherein the catheter comprises the ablation catheter.
